(19) United States
(12) Patent Application Publication
(10) Pub. No.: US 2006/0122703 A1
(43) Pub. Date: Jun. 8, 2006

(54) Intervertebral implant

(76) Inventors: Max Aebi, Bern (CH); Dominique Burkard, Gretzenbach (CH); Robert Frigg, Bettlach (CH); Beat Lechmann, Bettlach (CH); Robert Mathys JR., Bettlach (CH); Paul Pavlov, Nijmegen (NL)

Correspondence Address: JONES DAY, 222 EAST 41ST STREET, NEW YORK, NY, 10017-6702, US

(21) Appl. No.: 10/538,950

(22) PCT Filed: Dec. 17, 2002
(86) PCT No.: PCT/CH02/00707

§ 371 Date: Jun. 14, 2005

(51) Int. Cl. A61F 2/44

(52) U.S. Cl. 623/17.15

(57) ABSTRACT

An intervertebral implant (1), having an upper section (10) provided with a ventral side area (11), a dorsal side area (12), two lateral side areas (13,14), a top apposition surface (15) and a bottom surface (16); and a lower section (20) provided with a ventral side area (21), a dorsal side area (22), two lateral side areas (23,24), a bottom apposition surface (25) and a top surface (26). The two sections (10;20) are moveable in relation to each other by means of two joints (38;39) arranged between the two sections (10;20), the two joints (38;39) are realised by an upper joint section (31) connected to the upper section (10), a central joint section (32) and a joint section (33) connected with the lower section (20). One of the external joint sections (31;33) comprises at least one concave sliding surface (58) rotation-symmetrical with regard to a swivel axle (3;4); and the other of the external joint sections (31;33) comprises at least one convex sliding surface (55) rotation-symmetrical with regard to the other swivel axle (3;4).

The invention relates to an intervertebral implant according to the generic term of Patent claim 1 and to a process for the replacement of a defect, natural intervertebral disk by an intervertebral implant according to Patent claim 19.

After removal of a damaged, natural intervertebral disk or a damaged nucleus pulposus of an intervertebral disk, implants or prostheses are inserted into the intervertebral space of two neighbouring vertebral bodies. This suggests the idea of restoring the situation as much as possible to a natural state, i.e. specifically to restore the original height of the intervertebral disk and thus the original distance between the two neighbouring vertebral bodies. Furthermore, the patient should be able to carry out movements of the neighbouring vertebral bodies relative to each other in the natural way, thereby incurring as little obstruction as possible. This essential feature of this system is its ability to retain the freedom of movement in forward/reverse inclination, i.e. flexion and extension of the vertebral bodies, and in lateral bending of the vertebral bodies within the natural limits. The natural sinews and muscles along the spinal column are in general left intact so that they further stabilise the movements of a mechanical intervertebral disk prosthesis.

A characteristic intervertebral disk endoprosthesis is state of the art from DE-A 35 29 761 BÜTTNER. This known intervertebral disk endoprosthesis basically consists of two symmetric closing plates with concave sliding surfaces facing each other, and each having an external surface for laying on the base plate, or the cover plate of the adjoining vertebral body, and a distance piece positioned between the closing plates with convex sliding surfaces arranged complementary to the concave sliding surfaces on the closing plates. The sliding surfaces are designed in one embodiment as section surfaces of a cylinder coat area, wherein the sliding surfaces arranged on the two closing plates are provided complementary to each of the adjoining sliding surfaces at the distance piece, and two complementary sliding surfaces form the articulation surfaces, which can be moved towards each other, of a joint element rotating around a swivel axle. The joint comprises an upper and a lower joint element, each of which has one swivel axle. The two swivel axles are set at 90° to each other. The disadvantages of this known intervertebral disk endoprosthesis is that

a) the arrangement of an intervertebral disk endoprosthesis with only one fulcrum does not take sufficient account of the overlaying swivel movements transferred by the natural intervertebral disk, specifically in the case of anterior-posterior and in lateral flexion, which in the natural intervertebral disk are independent of each other;

b) the verterbral joint is put under strain by swivel movements, specifically with translation in the anterior-posterior direction (face joint), which could cause pain for the patient;

c) disadvantageous friction forces are generated by two articulating surfaces sliding on each other. This also leads to wear on the surfaces, including also abrasion and resistance in movement of the joint elements. There is also the risk of the “stick slip” effect;

d) a mechanical intervertebral disk prosthesis can scarcely prevent the further degeneration of the affected movement segments. Restoration of the original freedom of movement significantly reduces pain, with the resulting improvement to the patient's quality of life. A review of treatment will, however, have to be undertaken if pain recommences. This will normally involve complete removal of an intervertebral disk prosthesis of the standard model and a stiffening of the movement segment. This operation represents extreme discomfort and strain on the patient; and

e) the form of contact areas to the neighbouring vertebral bodies is generally not taken into account. The conventional types of intervertebral disk prosthesis implants have flat contact areas, which are often supplemented with keel-type elevations.

The invention is intended to remedy this situation. The invention is based on the task of creating an intervertebral implant that allows only swivel movements around certain swivel axes and does not permit any translation movements of the vertebral bodies.

The invention solves the task with an intervertebral implant that has the features of claim 1 and with a process for replacing a defect, natural intervertebral disk by an intervertebral implant, comprising the steps of claim 19.

The advantages achieved by the invention can generally be seen in that with the intervertebral implant according to the invention
the swivel movements in anterior-posterior and lateral direction are independent of each other;
no translation movements of the vertebral bodies cannot take place, which relieves strain on the face joints;
the swivel axles take account of the anatomic situation.

In a preferred embodiment of the intervertebral implant according to the invention, sliding surfaces are arranged as part sections of circular cylinder coat areas. Instead of part sections made of circular cylinder coat areas, part sections are also possible made of other rotation-symmetrical cylinder coat areas, for example cone coat areas.

In another embodiment of the intervertebral implant according to the invention, the lower joint section comprises, for example, at least one lower concave sliding surface with rotation-symmetry with regard to the first swivel axle and the central joint section at least one lower convex sliding surface complementary to the lower concave sliding surface. The upper joint section comprises at least one upper convex sliding surface with rotation-symmetry with regard to the second swivel axle and the central joint section at least one upper concave sliding surface complementary to the upper convex sliding surface. A reverse of the two pairs of sliding surfaces so that the upper joint section comprises at least upper concave sliding surface with rotation-symmetry with regard to the first swivel axle and the central joint section comprises at least one upper convex sliding surface complementary to the upper concave sliding surface is also possible. In this case, the reversal shall also apply for the lower joint section, which then comprises at least one lower convex sliding surface with rotation-symmetry with regard to the second swivel axle, whereas in this case the central joint section comprises at least one lower concave sliding surface complementary with regard to the lower convex sliding surface. This arrangement of the joint, i.e. in a way that the central joint section is provided with a convex sliding surface and a concave sliding surface with regard to the central axle axially opposite, and the external joint sections being arranged complementarily, allows a minimal structural height of the intervertebral implant to be achieved.

Due to the different positions of the natural swivel axles in the different intervertebral disk spaces along the spinal column the arrangement of the swivel axles can be skewed or intersecting.

The material combinations most suitable for the sections of the intervertebral implant fitted with sliding surfaces are generally metal-metal, metal-ceramic or metal-plastic combinations. Metal alloys with or without iron content are preferable for the metal part, while AlOₙ and ZrOₙ are the preferred choices for the ceramic material in the combination. High-molecular thermoplastics will be preferably used as plastics used for implants, although the material PEEK can also be used.

In a further embodiment of the intervertebral implant according to the invention, a means can be attached to the two sections from the ventral side areas which fixes the two sections ventral at a specific distance relative to each other. This measure provides the advantage that the two sections for insertion into the intervertebral space can be brought to a position with fixed height and can be moved around the joint after insertion into the intervertebral space and can be placed on the base or cover plate of the adjoining vertebral body.

In a further embodiment of the intervertebral implant according to the invention, the means allows temporary blocking of the mobility of the two sections around the joint. This measure provides the advantage that the joint integrated in the intervertebral space can be blocked by a minimum invasive operation. This is particularly advantageous in cases where the patient suffers from post-operative pain, i.e. where degeneration of the affected spinal column segment continues and the surgeon is considering a fusion of the affected vertebra. The means can preferably be attached to the two ventral side areas of the two sections. With this subsequent, secondary blocking of the mobility of the two sections around the joint, the intervertebral implant is stiffened and transferred to an arthrodesis implant (fusion cage).

In a further embodiment of the intervertebral implant according to the invention, the means comprises an insert, which can be placed into each depression on the surfaces of the upper and lower section opposite each other. These depressions are preferably provided as dovetail guides that are open on the ventral side areas, so that the ends of the insert arranged complementary to the dovetail guides can be inserted from ventral into the dovetail guides. This provides the advantage that the mobility of the two sections around the joint is blocked due to the positioning of the insert. The rigidity of the blocking can be increased when the dovetail guides are designed so that they are reduced is size towards the central axis of the intervertebral implant, which creates additional wedging of the insert in the dovetail guides.

In a further embodiment of the intervertebral implant according to the invention, the two sections are provided with drill holes for receiving the bone fixation means, specifically bone screws, wherein the drill holes are provided with longitudinal axes that stand perpendicular to the central axis. Preferably two drill holes will pass through one of the two sections from the ventral side area to the apposition surface. The longitudinal axes, if only an axial fixing of the intervertebral implant is provided, will then be able to stand only perpendicular to the central axis from a lateral perspective, or, if fixing of the intervertebral implant with stable angle is provided, will also from a lateral perspective diverge from the inner surfaces of the two sections against the apposition surfaces.

In a further embodiment of the intervertebral implant according to the invention, the drill holes for receiving the bone fixation means are provided with internal threads, which allows additional, rigid fixing of the bone fixation means in the two sections. The drill holes preferably have a conical shape so that a stronger fixing of the bone fixation means to each of the two sections can be achieved by the resulting conical thread connections between the internal threads and the external threads on the heads of the bone fixation means.

The apposition surfaces are preferably of convex shape and provided with a three-dimensional structure, preferably in the form of pyramid elevations. This arrangement of the apposition surfaces takes account of the anatomy of the vertebral body end plates.

The process according to the invention is intended primarily for replacing a defect, natural intervertebral disk by an intervertebral implant and comprises the following steps:
A) blocking of the joint(s) of an intervertebral implant by means of a special device placed in a certain position of the joint(s);
B) insertion of the intervertebral implant into the intervertebral space to be treated;
C) release and removal of the device inserted into the intervertebral implant for blocking the joint(s). Blocking the joint provides the advantage that the moveable sections with the external apposition surfaces can be inserted more easily into the intervertebral space to be treated.

In a further application of the process according to the invention, this comprises the subsequent blocking of the joint(s) on the implanted intervertebral implant by means of the device intended for blocking the joint(s). This provides the advantage that if the patient should suffer from post-operative pains or in case of a further degeneration of the movement segment, the joint(s) on the intervertebral implant are blocked post-operative by the insertion of the means intended for this purpose. This subsequent blocking can be achieved with an minimally invasive, preferably a laprascopic operation. The intervertebral implant then assumes the function of a cage, so that the affected movement segment of the spinal column can be stiffened.

The invention and refinements of the invention are described in more detail below on the basis of a partially schematic illustration of several embodiments.

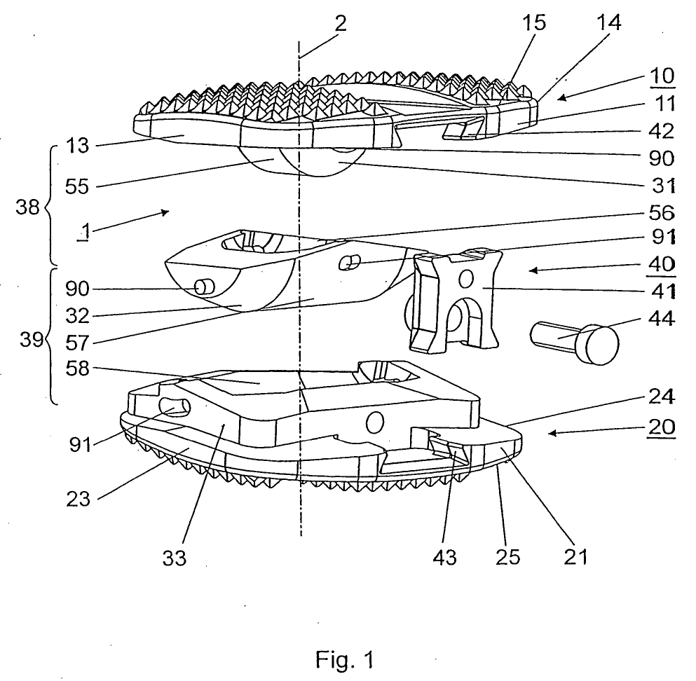

FIG. 1 shows an explosion diagram of one embodiment of the intervertebral implant according to the invention;

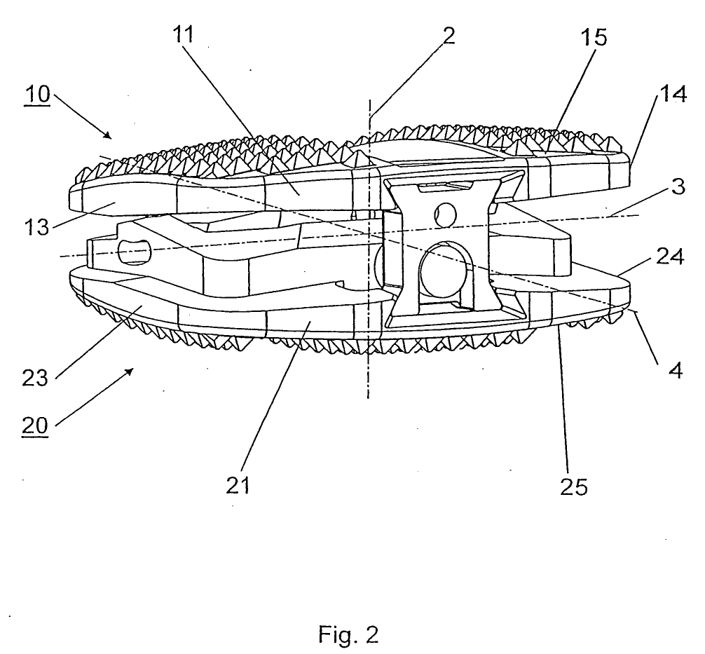

FIG. 2 shows a perspective view of the embodiment of the intervertebral implant according to the invention shown in FIG. 1 in assembled state;

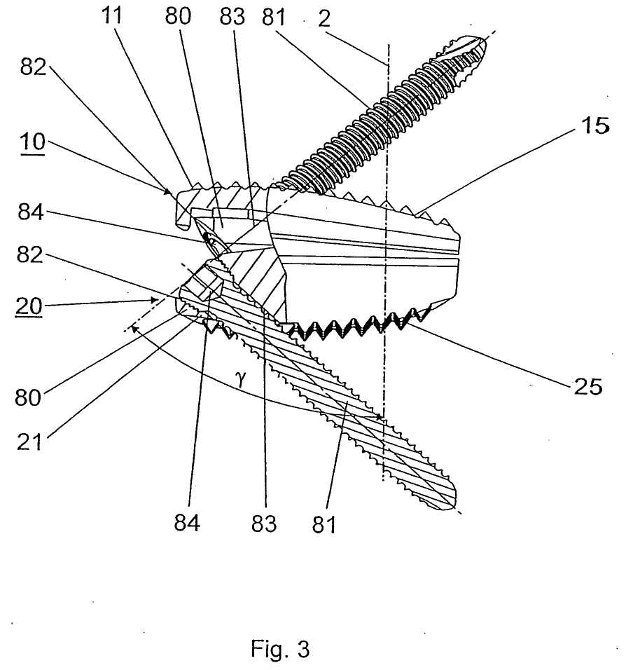

FIG. 3 shows a lateral view of a further embodiment of the intervertebral implant according to the invention; and

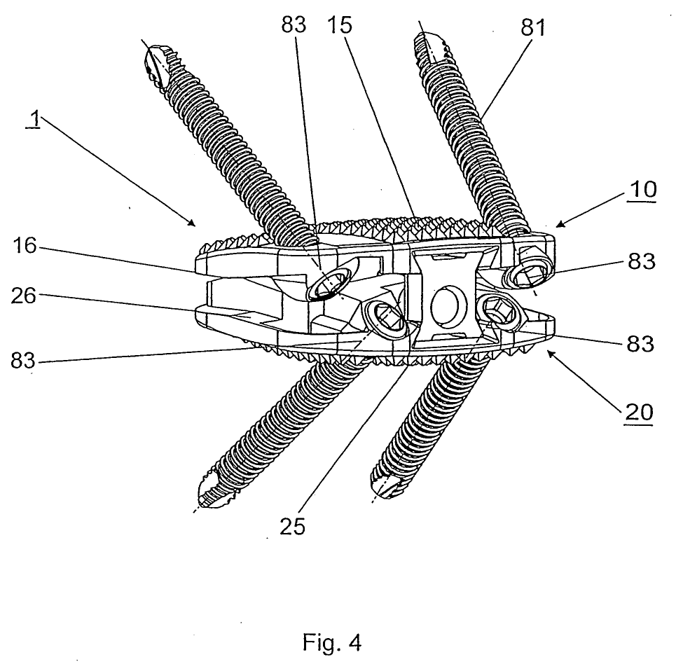

FIG. 4 shows a perspective view of the embodiment from ventral according to FIG. 3.

FIG. 1 and FIG. 2 show an embodiment of the intervertebral implant 1 according to the invention, which comprises an upper section 10 with a top apposition surface 15 arranged perpendicular to the central axis 2 for laying on the base plate of an adjoining vertebral body, a lower section 20 with a lower apposition surface 25 arranged perpendicular to the central axis 2 for laying on the cover plate of the adjoining vertebral body and two joints 38;39. The upper section 10 and the lower section 20 are connected with the joints 38;39 and moveable in relation to each other, whereby the mobility of the upper section 10 relative to the lower section 20 is restricted by a first swivel axle 3 arranged perpendicular to the central axis 2 within an angle range of ±10° to −6° and by a second swivel axle 4 arranged perpendicular to the central axis 2 and vertical to the first swivel axle 3 within an angle range of ±7°.

The two joints 38;39 are realised by three joint elements 31;32;33, of which the lower joint element 33 and the upper joint element 31 each form a joint 38;39 interacting with the central joint element 32. The two joints 38;39 are each provided with a swivel axle 3;4, wherein the swivel axles stand vertical to each other and vertical to the central axis 2. The lower joint 39 comprises as articulation surfaces a lower convex sliding surface 57 arranged on the central joint element 32 and coaxial to the first swivel axle 3, and a lower concave sliding surface 58 arranged on the lower joint element 33 complementary to the sliding surface 5. The upper joint 38 comprises as articulation areas an upper convex sliding surface 55 arranged on the upper joint section 31 and coaxial to the second swivel axle 4, together with a lower concave sliding surface 56 arranged on the central joint section 32 and complementary to the sliding surface 55. The sliding surfaces 55;56;57;58 are arranged as part sections of circular cylinder coat areas.

In addition, coaxial cams 90 are also attached to the upper and central joint section 31;32 axially terminal to the swivel axles 3;4, which are fitted with sliding action in oblong hole guides 91 in the lower joint element and in the central joint element 32.

Because of the cams 90 moving in the oblong hole guides 91, the swivel angles of the joint elements 31;32;33 around the swivel axles 3;4 are limited. In addition, the intervertebral implant 1 is held together by the cams 90 positioned in the oblong hole guides 91.

The mobility of the two sections 10;20 relative to each other can be blocked by the means 40 in a way that allows release. The means 40 comprises in the embodiment described here an insert 41 that can be slid in from the ventral side areas 11;21 of the two sections 10;20 perpendicular to the central axis 2 and parallel to the lateral side areas 13;14;23;24 of the two sections 10;20. The insert 41 is slid in two depressions 42;43, provided in the form of dovetail guides. The insert 41 is inserted from the ventral side areas 11;21 of the two sections 10;20 into the depressions 42;43 composed as dovetail guides and fitted to the lower section 20 by means of a screw 44. The insert 41 is furthermore arranged in the terminal state complementary to the depressions 42;43, so that the two sections 10;20 with fitted insert 41 are fixed relative to each other parallel to the central axis 2.

FIG. 3 and FIG. 4 illustrate an embodiment of the intervertebral implant 1 according to the invention, which differs from the embodiment illustrated in FIG. 1 and FIG. 2 only in that the two sections 10;20 also comprise drill holes 80 for receiving the bone fixation means 81, whereby the bone fixation means 80 is provided in this case as bone screws. The drill holes 80 are provided with longitudinal axes 83 that form an angle γ with the central axis 2. In addition, each two drill holes 80 run trough one of the two sections 10;20 from the ventral side area 11;21 to the apposition surface 15;25. The longitudinal axes 83 of the drill holes 80 are standing perpendicular to the central axis 2 from both a lateral perspective (FIG. 3) and from a ventral perspective (FIG. 4). The drill holes 80 are furthermore provided in conical design and tapering towards the apposition surfaces 15;25 and provided with internal threads 82 that are used for screwing reception of the screw heads 84 of the bone fixation device 81 realised here in the form of bone screws and provided with complementary external threads.

1. An intervertebral implant (1), specifically an artificial intervertebral disk, comprising a central axis (2), an upper section (10), suitable for laying onto the base plate of a vertebral body lying above and a lower section (20) suitable for laying onto the cover plate of a vertebral body lying below, wherein
A) the upper section (10) is provided with a ventral side area (11), a dorsal side area (12), two lateral side areas (13, 14), a top apposition surface (15) and a bottom surface (16);
B) the lower section (20) is provided with a ventral side area (21), a dorsal side area (22), two lateral side areas (23,24), a bottom apposition surface (25) and a top surface (26); wherein
C) the two sections (10;20) moveable in relation to each other by means of two joints (38;39) arranged between the two sections (10;20),
D) each of the joints (38;39) is provided with a swivel axle (3;4) and the two swivel axles (3;4) are arranged perpendicular to each other;
E) the two joints (38;39) are realised by means of an upper joint element (31) connected with the upper section (10), a central joint element (32) and a joint element (33) connected with the lower section (20);
F) one of the external joint sections (31;33) comprises at least one concave sliding surface (58) rotation-symmetrical with regard to a swivel axle (3;4); and
G) the central joint section (32) comprises at least one convex sliding surface (57) complementary to this concave sliding surface (58), wherein
H) the other of the external joint sections (31;33) comprises at least one convex sliding surface (55) rotation-symmetrical with regard to the other swivel axle (3;4); and
I) the central joint section (32) comprises at least one concave sliding surface (56) complementary to this convex sliding surface (55)
K) the sliding surfaces (55;56;57;58) are configured as partial surface areas of circular cylindrical or circular conical surface areas; and
L) the swivel axes (3;4) are arranged skewed to each other.

2. The intervertebral implant (1) according to claim 1, wherein the lower joint element (33) comprises at least one lower concave sliding surface (58) rotation-symmetrical with regard to the first swivel axle (3) and the central joint section (32) at least one lower convex sliding surface (57) complementary to the lower concave sliding surface (58).

3. The intervertebral implant (1) according to claim 1, wherein the upper joint element (31) comprises at least one upper convex sliding surface (55) rotation-symmetrical with regard to the second swivel axle (4) and the central joint section (32) at least one upper concave sliding surface (56) complementary to the upper convex sliding surface (55).

4.-6. (canceled)

7. The intervertebral implant (1) according to claim 1, wherein a means (40) is provided that keeps the two sections (10;20), measured at their ventral side areas (11;21), at a fixed distance from each other.

8. The intervertebral implant (1) according to claim 1, wherein a means (40) is provided that is suitable for causing temporary blocking of the mobility of the two sections (10,20) around the joints (38;39).

9. The intervertebral implant (1) according to claim 7, wherein the means (40) can be attached to the two ventral side areas (11,21) of the two sections (10;20).

10. The intervertebral implant (1) according to claim 8, wherein the means (40) include an insert (41) with a lower end (45) and an upper end (46) and a depression (42;43) in the surfaces (16;26) at each of the two sections (10;20), which are open on the ventral side areas (11;21), and that the insert (41) with its ends (45;46) can be inserted into each of the depressions (42;43).

11. The intervertebral implant (1) according to claim 10, wherein the depressions (42;43) are dovetail guides and the ends (45;46) on the insert (41) are arranged complementary to these dovetail guides.

12. The intervertebral implant (1) according to claim 11, wherein the dovetail guides are tapered from the ventral side areas (11;21) towards the dorsal side areas (12;22).

13. The intervertebral implant (1) according to claim 1, wherein the upper and the lower section (10;20) each comprises at least two drill holes (80) running through from the ventral side areas (11;21) to the apposition surfaces (15;25) with longitudinal axes (83) for receiving bone fixation devices (81).

14. The intervertebral implant (1) according to claim 13, wherein the longitudinal axes (83) of the drill holes (80) make an angle γ with the central axis (2).

15. The intervertebral implant (1) according to claim 14, wherein the angle γ lies in a range of between 20° and 65°.

16. The intervertebral implant (1) according to claim 13, wherein the longitudinal axes (83) of the drill holes (80) as seen from the ventral side areas (11;21) diverge from the inner surfaces (16;26) against the apposition surfaces (15;25).

17. The intervertebral implant (1) according to claim 13, wherein the drill holes (80) are conically tapered towards the apposition surfaces (15;25).

18. The intervertebral implant (1) according to claim 13, wherein the drill holes (80) are provided with an internal thread (82).

19. A process for the replacement of a defect, natural intervertebral disk characterised by an intervertebral implant, comprising the steps:
A) blocking of the joint(s) (38;39) of an intervertebral implant (1) through the special means (40) in a certain position of the joint(s) (38;39);
B) insertion of the intervertebral implant (1) into the intervertebral space to be treated; and
C) release and removal of the device (40) inserted into the intervertebral implant (1) for blocking the joint(s) (38;39).

20. The process according to claim 19, additionally comprising the step of the subsequent blocking of the joint(s) (38;39) on the implanted intervertebral implant (1) through the means (40).
